(19) United States
(12) Patent Application Publication
(10) Pub. No.: US 2017/0249424 A1
(43) Pub. Date: Aug. 31, 2017

(54) METHOD FOR SHARING PATIENT PREGNANCY DATA DURING ULTRASOUND

(71) Applicant: SAMSUNG ELETRÔNICA DA AMAZÔNIA LTDA., Campinas (BR)

(72) Inventors: Marcelo AUGUSTO DI GRANDI NERY, Campinas (BR); Gustavo Kaneblai Martins Costa, Campinas (BR); Rodrigo Jose Tobias, Campinas (BR); Renata Zilse Pereira Borges, Campinas (BR); Myung Hwa Baldini, Campinas (BR); Viviane Ortiz Franco, Campinas (BR); Michel Bez Fontana, Campinas (BR); Roberta Mayumi Matsunaga, Campinas (BR); Alexandre Barbosa Silveira, Campinas (BR)

(73) Assignee: SAMSUNG ELETRÔNICA DA AMAZÔNIA LTDA., Campinas (BR)

(21) Appl. No.: 15/282,387

(22) Filed: Sep. 30, 2016

(30) Foreign Application Priority Data
Feb. 25, 2016 (BR) 10 2016 004181 3

(51) Int. Cl. G06F 19/00; A61B 8/00; A61B 8/08; G06F 21/62; G06Q 50/00

(52) U.S. Cl. CPC G06F 19/322; G06F 21/6245; G06Q 50/01; G06F 19/321; A61B 8/0866; A61B 8/565

(57) ABSTRACT

A method for sharing patient pregnancy data during ultrasound examination, including: capturing relevant pregnancy data by means of an ultrasound machine by the doctor/sonographer without exam interruption; storing pregnant relevant data captured/collected from the pregnant into a local machine; and transmitting the compressed relevant data sent by the local machine to the cloud service, assigned with a unique patient identification; and allowing access of the smart device to a cloud service assigned with a unique patient identification through a web service.

### FIELD OF THE INVENTION

The present invention refers to a method for sharing collectable data about the patient's pregnancy during ultrasound. More specifically, the present invention refers to a method for allowing the users to have a single experience in the pregnancy process, wherein ultrasound exams/images and others information/data (e.g. heart beat sound, etc.) referring to the pregnancy can be shared in an automatic way, through the ultrasound machine and a specific application, allowing friends, family and others people to also experience the pregnancy and share each moment of it.

### BACKGROUND OF THE INVENTION

One of the most memorable moments in the life of a person or family is the born of a child. Before the child is born, there are a lot of special moments during the pregnancy that are also important, and in most of cases these moments are not registered due to a lot of reasons like: lack of time, forgetfulness and sometimes there are no way to register that specific moment or feeling. Sometimes, that special moment of the pregnancy cannot be shared with the father, due physical restrictions or others reasons, takes too long to be shared, that special moment may not have the same values and feelings as if it was instantly shared.

One of these special moments is the ultrasound examination. The ultrasound examination is considered by many as the opportunity to “meet” the baby. Therefore, many families request the recording of this exam. Some clinics offers to record the exam images/video on a DVD or USB-Drive which are not appropriate for easy sharing on social networks, leading some families to use their mobile phones to record the ultrasound screen during the examination.

Some documents from the prior art aim to solve the problems related to the sharing of ultrasound exams, as it will be further described.

Patent document U.S. Pat. No. 6,440,072 B1, titled “Medical Diagnostic Ultrasound Imaging System and Method for Transferring Ultrasound Examination Data to a Portable Computing Device”, published on Aug. 27, 20102, by ACUSON CORPORATION, describes a medical diagnostic ultrasound imaging system and method for transferring ultrasound examination data to a portable computing device. In one preferred embodiment of document U.S. Pat. No. 6,440,072 B1, ultrasound examination data are transferred from a medical diagnostic ultrasound imaging system to a portable computing device, such as a personal digital assistant. The ultrasound examination data can be viewed on the portable computing device or further transferred to a review station or another portable computing device for review. In some preferred embodiments of document U.S. Pat. No. 6,440,072 B1, the examination data is converted from a form readable by the ultrasound system to a form readable by the portable computing device or review station. The ultrasound examination data can be transferred using a wired connection or using wireless technology, such as an infrared communications link. The preferred embodiments of document U.S. Pat. No. 6,440,072 B1 can also be used with other medical acquisition devices and medical examination data. Examination data can also be transferred from medical networks. The document U.S. Pat. No. 6,440,072 B1 differs from the method of the present invention due to present just a solution to execute ultrasound files in smart devices in a readable extension. The present invention goes beyond the way of executing a file, because it provides an experience of sharing image and content of a pregnancy stored in a cloud, and through a dedicated web service, it's possible to share with people these content.

Patent document US 2005/0215900 A1, titled: “System and Method for Providing Ultrasound Images of a Target Object Through a Wireless Communication Network”, published on Sep. 29, 2005, by MEDISON CO., LTD, describes a system for providing an ultrasound image of a target object, including: ultrasound diagnostic equipment for acquiring ultrasound data from the target object; an image converter for converting ultrasound data into mobile ultrasound data having a data format, which is suitable for transmission through a wireless communication network; and a mobile server for transmitting the mobile ultrasound data through the wireless communication network. The matter presented in document US 2005/0215900 A1 differs from the present invention because it discloses a solution for 2D and 3D ultrasound images being recognized in smart devices and heart beat sound, but it doesn't bring a solution of sharing pictures/files of ultrasound exams with people. Neither it is possible for these people and patient to upload and to share information about the pregnancy through a dedicated web service (cloud).

Patent document WO 2014/041448 A1, titled: “Mobile 3d Wireless Ultrasound Image Acquisition Device and Ultrasound Imaging System”, published on Mar. 20, 2014, by MCKEE DUNN POLAND, describes an ultrasound image acquisition device for use together with a mobile console to form an ultrasound imaging system. Further, the disclosed matter of document WO 2014/041448 A1 relates to an ultrasound imaging system for providing an image of a volume, for example an anatomical view within a body of a patient. Document WO 2014/041448 A1 differs from the method of the present invention because it proposes a solution for ultrasound images through a device to be used together with a mobile console to form ultrasound images. The method of the present invention gets images/videos and sounds automatically from ultrasound exams, and then preprocesses, compacts and provides this information for a cloud, allowing the patient to share these content with friends and family.

Patent document US 2015/038844 A1, titled: “Portable Ultrasound System Comprising Ultrasound Front-End Directly Connected to a Mobile Device”, published on Feb. 5, 2015, by Travis Blalock et al., describes a portable ultrasound imaging system which includes: a mobile computing device; a detachable front end component configured for attachment to and communication with the mobile computing device, and configured to transmit and receive ultrasound signals; and programming, when installed on the mobile computing device, being executable by the mobile computing device to cause the mobile computing device to send signals to the front end component causing the front end component to transmit the ultrasound signals, and to receive signals from the front end component, and process the receive signal and display an ultrasound image resulting from the processing. The front-end component is configured to be directly joined with the mobile computing device and directly connected, without the use of an external wire or cable. Document US 2015/038844 A1 differs from method of the present invention because it is related to a component that is attached to a mobile computing device to send signals and receive information and ultrasound images, with the possibility of executing these files in a smart device. The method of the present invention has another approach for ultrasound images and shares images using a dedicated web service with a dedicated system developed for this use.

### SUMMARY OF THE INVENTION

The present invention refers to a method and system to provide a new experience related to capture and consumption of collectable data related to pregnancy during ultrasound exams.

The main objectives of the present invention is:

to provide a new experience for doctors/sonographers, pregnant women, family and friends;

to capture and collect all relevant data related to the pregnancy journey;

to optimize the ultrasound exams;

to provide the pregnant and their family/friends with a possibility of sharing information mentioned above (pictures, videos, sounds and others information available during the exam) through a specific web page in real time; and

allow a person to follow the exam in real time, even if this person is not physically in the exam room (cameras, microphones, etc.).

A preferred embodiment of the method of the present invention consists of capturing data (images, video, audio, etc.) during the ultrasound exam, temporarily store, compress and pre-process these data into a local machine associating an unique client/patient identification (email, social security number, etc.), transmit and store these data into a cloud service which hosts a web service responsible for allowing connected internet devices (mobile phones, tablets, computers, etc.) to access, manage, share, etc., the data captured during the ultrasound exam.

More specifically, the objects and advantages described above are achieved by a method for sharing collectable data about the pregnancy of a patient during ultrasound examination which comprises the steps of:

capturing relevant pregnancy data by means of an ultrasound machine by the doctor/sonographer without exam interruption;

storing pregnant's relevant data captured/collected from the pregnant into a local machine; and

transmitting the compressed relevant data sent by the local machine to the cloud service, assigned with a unique patient identification;

allowing access of the smart device to a cloud service assigned with a unique patient identification through a web service.

An example system to support the method of the present invention is described below. Such system is composed by ultrasound machines responsible for collecting data and transmitting the data to a local machine/computer. The local machine/computer is responsible to store, pre-process and compact/compress received data and then transmitting the data to a cloud service in a proper moment (with good internet access, all data properly processed, exam properly finished, etc.). The cloud service is responsible to store, protect accesses, make available and allow data management required by connected devices. The devices connected to the cloud service are capable to retrieve the allowed data, share it with others users and add more data to the cloud.

Such as disclosed in the present invention, the proposed solution provides an innovative experience for pregnant and their friends/families (especially those which, in most of cases, cannot be close due to any reason), so that it is possible to share all relevant data and moments during the pregnancy journey, without the need of undesirable interruptions during the exam, which results in optimizing the doctor's time and the use of the ultrasound machine. Sharing of information may be in synchronous mode (i.e., in real time, for example, so that people who are not close to the exam can accompany the exam at the moment it happens, with streaming or video conference) or in asynchronous mode (i.e., storing relevant data into a cloud service for further access and sharing by the pregnant, her doctor, family and friends).

### BRIEF DESCRIPTION OF DRAWINGS

The objectives and advantages of the present invention will become clearer through the following detailed description of an example and non-limitative embodiment together with the attached figures, wherein:

FIG. 1 discloses a general view of the traditional method from the prior art to perform ultrasound images.

FIG. 2 discloses a general view of freely adapted method from the prior art to perform ultrasound images.

FIG. 3 discloses a flowchart according to a preferred embodiment of the method of the present invention.

FIG. 4 discloses a general view of an example system which implements a preferred embodiment of the method of the present invention.

FIG. 5 discloses a preferred embodiment of the method of the present invention.

### DETAILED DESCRIPTION OF THE INVENTION

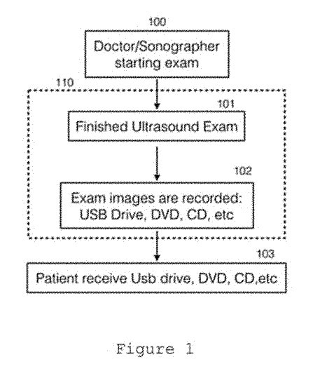

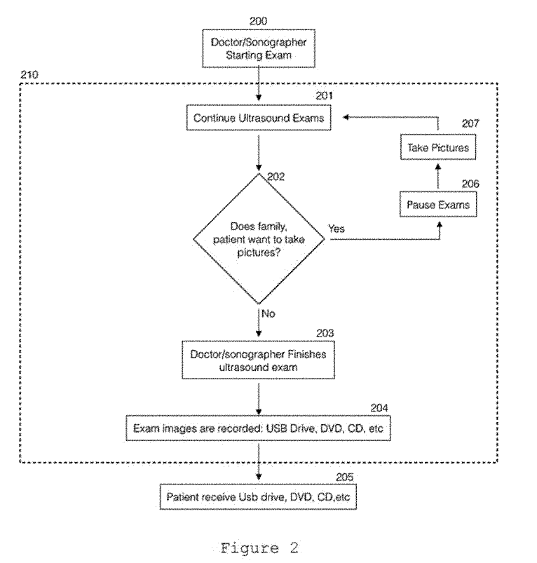

As mentioned above, the ultrasound exam is considered by many as the opportunity to “meet” the baby. Therefore, many families request the recording of this exam. However, the current methods are not appropriated to readily and easily share the ultrasound exam images (FIG. 1), what in some cases lead people to use their mobile phones to record the ultrasound screen during the exam (FIG. 2), which can result in slower exams (as the doctor has to interrupt the exam so that the patient/companion take a picture or record a video of the ultrasound screen).

For illustration purposes, FIG. 1 presents the traditional method to perform ultrasound exams and the consumption of this information. The doctor or sonographer 100 performs the exam 101 on the patient. Since the exam is finished, according to request by patient 103, it can be recorded 102 in USB drive, DVD, CD, etc. However, the method 110 of performing ultrasound exams does not provide the social needs of easily sharing the ultrasound images with friends and family.

FIG. 2 presents the current/traditional method for ultrasound exams, freely adapted to support the sharing of pictures/videos on social networks. The doctor or sonographer 200 starts to perform the exam 201. Nowadays, it is very common the patient to take pictures of the screen machine in order to record and share these pictures with friends and family. When this event does not happen 202, the exams are finished 203 and recorded 204. Because of the necessity to share pictures with friends and family, the exams are interrupted 206 to take pictures 207 and register that moment. When the exam is completely performed, the ultrasound images 203 are stored 204 in a USB, DVD or other media. The patient 205 is used to share the pictures of the exam taken by external device (for example, photographic camera of his smartphone) with friends and social networks.

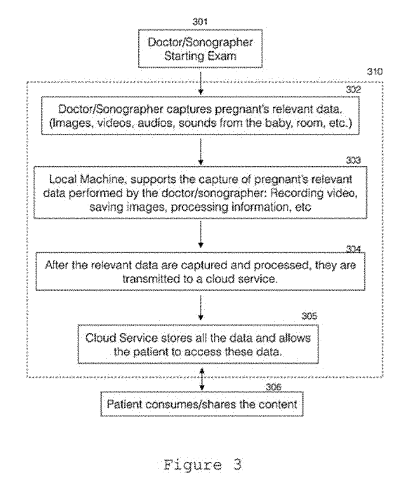

FIG. 3 presents a preferred embodiment of the method of the present invention for capturing and using ultrasound collectable data and images, in order to solve or mitigate the drawbacks of the existing solutions, as reported on the paragraphs above. The method 310 describes the steps wherein the doctor or sonographer 301 performs the ultrasound exam capturing all relevant data 302 (ultrasound images/videos, room camera data, heart beats from the mother and the baby, etc.), without interrupting the doctor/sonographer 301 during the procedure. During the exam, the local machine (e.g. a computer/laptop) 303 supports the capture and storage of pregnant's relevant data, that will be further pre-processed and compressed to be transmitted to the cloud service 304, in a proper moment (e.g. after the exam completely finishes). The cloud service 304 is used to store the relevant data (pre-processed, compressed and sent by local machine 303) and make it available through a web service. Patient (or any other interested person) can access the cloud service 305 through devices (smartphones, PCs, tablets, smart TVs, etc.) to access the data related to the patient, and then sharing with friends and family and also upload images, videos, etc. 306 complementarily (or not necessarily related) to ultrasound exam.

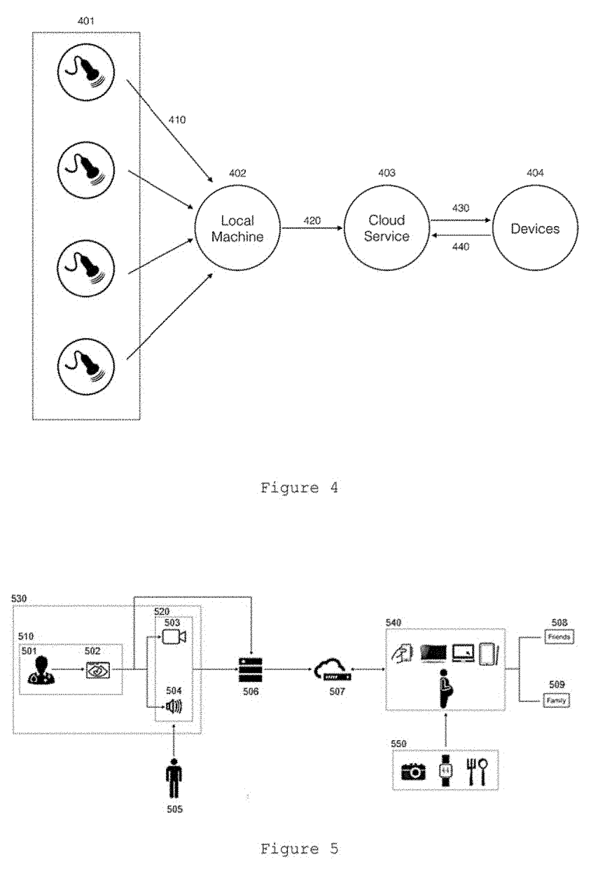

FIG. 4 illustrates a general view of the example system which implements the proposed method of the present invention. The system is composed by ultrasound machines 401, which are handled by the doctor/sonographer in order to collect relevant data from exams (according to step 302 of the method of the present invention 310). All data collected by ultrasound machines 401 (and environment sensors, e.g. cameras, microphones, etc.) during the exam is sent 410 to the local machine 402 in order to pre-process, compress and temporarily store relevant all data collected (according to step 303 of the method of the present invention 310). When all data exams are finished and properly pre-processed, the local machine 402 will send 420 all data to the cloud service 403 (according to step 304 of the method of the present invention 310). The cloud service 403 stores, manages, protects/authenticates accesses and makes data available 430 (according to step 305 of the proposed method 310). By using smart devices 404 (like laptops, smartphones, wearable devices, computers, smart TVs, tablets and others with Internet access), the patient (or any other interested person) can retrieve the available data in the cloud service 403, with the possibility to send the data to others users or share with family and friends on social networks. Optionally (if necessary), patient may also include/upload 440 new data/information in the cloud service 403 (according to step 306 of the method of the present invention 310).

The cloud service 403 allows: invite people to share information/data stored in the cloud, allowing these people “monitor or interact” with the pregnancy through the information/data shared; include new or complementary information/data of the pregnancy; people invited to share information/data of the pregnancy can include new information on the cloud service 403; conversation, chat, messages and data/information posts through a timeline; to follow another pregnancy like “Follower”.

For a better understanding of the method of the present invention, FIG. 5 presents an illustration of an embodiment of the present invention and its usage/operation. In an exam room 530, the doctor/sonographer 501 performs an ultrasound exam 510 (according to the proposed method 310) extracting data and ultrasound images and videos 502. The exam room 530 is also equipped with a set of devices and sensors (e.g. video camera 503 and a microphone/audio system 504) which can be used to collect environmental data (e.g. images from the pregnant woman and the doctor, sounds/voices, etc.) and allow an absent person 505 (e.g. the father or family members that could not be present on that exam) to remotely follow the exam in real time (e.g. by video conference through the Internet). The collected image/data 502, sounds 504 and videos 503 of the exam are transmitted to the local machine 506, which pre-processes, temporarily stores and compresses all data provided during the ultrasound exam. All data is associated to a unique patient identification (email, social security number, etc.). After the local machine 506 pre-processed, compressed and temporarily stored the data, the data is then transmitted to the cloud service 507 (assigned to the patient identification), which will keep the information stored and safe (with authentication process to make available only the data uniquely associated to a specific patient).

The procedure to send directly the ultrasound images/videos/sounds 502, 503, 504 to a local machine 506 avoids overloading the ultrasound machine, since the main function of this machine is to provide ultrasound images 502 for exams. The process of sending information from the local machine 506 to cloud service 507 also avoid the local machine overload, since the main function of the local serve machine is just providing the images/data processing to be stored in the cloud.

Through smart devices 540 (like laptops, smartphones, wearable devices, computers, smart TVs, tablets and others with internet access), the pregnant/patient directly connects to the cloud service 507, using her patient identification (to authenticate herself) and a dedicated application (web service) developed for this purpose, managing all data stored in it.

The patient can also include external information 550 to the cloud service 507 like: photos, health information (exercises, diet), etc. through her smart device 540 connected to Internet and authenticated to the cloud service 507.

The patient has the possibility to share all information stored in the cloud service 507 through the dedicated application (web service) with friends 508 or family 509. The process to share information is simple. There is an invite done by the patient to the people that will receive all information. The people invited by the patient can follow, access, add information about the pregnancy with comments and communicate through a timeline (in the same manner as a “social network”).

Optionally, all data (images, audio/sounds, videos, comments, etc. recorded by the patient and “followers”) stored in the cloud service 507 can be compiled and provide a history (timeline) of all the pregnancy journey.

Although the present invention has been described in connection with certain preferred embodiment, it should be understood that it is not intended to limit the invention to that particular embodiment. Otherwise, it is intended to cover all alternatives, modifications and equivalents possible within the spirit and scope of the invention as defined by the following claims.

1. A method for sharing patient pregnancy data during ultrasound examination comprising:
capturing relevant pregnancy data by means of an ultrasound machine by the doctor/sonographer without exam interruption;
storing pregnant relevant data captured/collected from the pregnant into a local machine;
transmitting the compressed relevant data sent by the local machine to the cloud service, assigned with a unique patient identification; and
allowing access of the smart device to a cloud service assigned with a unique patient identification through a web service.

2. The method according to claim 1, wherein the storing pregnant relevant data comprises the compression of the data captured by the local machine.

3. The method according to claim 1, further comprises performing upload of complementary data through web service.

4. The method according to claim 3, wherein the performing the upload comprises establishing conversation, chat, messages and data/information posts through a timeline.

5. The method according to claim 1, wherein the allowing access to smart devices comprises allowing access to laptops, smartphones, PCS, tablets, smart TVs.

6. The method according to claim 1, wherein the cloud service allows an absent person to remotely follow the examination in real time.
